(19) United States
(12) Patent Application Publication
(10) Pub. No.: US 2018/0228511 A1
(43) Pub. Date: Aug. 16, 2018

(54) Modular Autoclavable Introducer for Endoscope

(71) Applicant: Apurba Mukherjee, Beverly Hills, CA (US)

(72) Inventor: Apurba Mukherjee, Beverly Hills, CA (US)

(21) Appl. No.: 15/431,028

(22) Filed: Feb. 13, 2017

(51) Int. Cl. A61B 17/34; A61M 13/00; A61B 1/00; A61B 1/313

(52) U.S. Cl. CPC A61B 17/3462; A61M 13/003; A61B 17/3474; A61B 2017/00535; A61B 1/00154; A61B 1/3132; A61B 17/3423

(57) ABSTRACT

An endoscopic or laparoscopic introducer, a medical device to assist the surgeon with the insertion, maneuvering, and removal of an endoscope, laparoscope, biopsy device, or medical device from a patient's body during endoscopic or laparoscopic surgery. The advancement relates to an introducer and specifically to an autoclavable modular introducer. Introducer is modular and is capable of attachment to numerous types of fittings and thereby capable of coupling and decoupling to numerous types of endoscopes, cannulas, insufflation flow sources, biopsy devices, and other medical devices. Introducer is autoclavable in that it is capable of sterilization by autoclave without incurring deterioration or degradation. This invention incorporates a biopsy port in the modular introducer system for the surgeon to extract cells and tissues for biopsy procedure.

### BACKGROUND OF THE INVENTION

### 1. Field of the Invention

The present invention relates to an endoscopic or laparoscopic introducer, which is a medical device that assists the surgeon with the insertion, maneuvering, and removal of an endoscope, laparoscope, biopsy device, or medical device from a patient's body during endoscopic or laparoscopic surgery. An endoscope or laparoscope is used by a surgeon to visually inspect small lesions or clusters of cells or an interior organ of a patient, where visual inspection is required for surgery. Endoscopic surgery is surgery performed through a small incision or natural opening in the patient's body where endoscope and introducer are inserted in tandem through the small incision or natural opening. This invention deals with an introducer and specifically to an autoclavable modular introducer. Introducer is modular in that it is capable of attachment to numerous types of fittings and thereby capable of coupling and decoupling to numerous types of endoscopes, cannulas, insufflation flow sources, biopsy devices, and other medical devices. Introducer is autoclavable in that it is capable of sterilization by autoclave without incurring deterioration or degradation.

### 2. Description of Related Art

Endoscopic or laparoscopic surgery is minimally invasive because it is performed through one or more small incisions or natural openings in the body. Typically, prior to or during the introduction of the endoscope, biopsy device, or other surgical object into the patient's body, an introducer pumps insufflation gasses or liquids into the small incision or natural opening that are used to enlarge the area surrounding the target surgical site in order allow for easier insertion, better visual inspection, and more work area for the surgeon. An introducer performs such tasks as follows:

An introducer comprises a hub or rigid hollow vessel capable of containing the pressurized flow of gas or liquid there through a long rigid cannula or tube at its distal end and connected to an endoscope port and an insufflation port on its proximal end. Insufflation port is typically coupled to a flow source of gas or liquid that forces the pressurized flow of gas or liquid into and through the hub, into the cannula, and out the distal end of the cannula. With this flow of gas or liquid occurring, the distal end of the cannula is inserted into the patient's body to enlarge the area of the patient's body. The flow of gas or liquid and ensuing enlargement allows for much easier insertion, navigation, maneuverability, and extraction of an endoscope. The endoscope is inserted into the endoscope port, through the hub, through the cannula, and then out of the distal end of the cannula. In this configuration, with the flow of insufflation gas or liquid, there is a “jacket” of gas or liquid flowing around the outside of the endoscope that allows for much easier insertion, movement, and withdrawal of the endoscope. The flow of gas or liquid through the distal end of the cannula is small in degree and is typically about a drop every few seconds or so.

The distal tip of the endoscope must protrude through the distal tip of the cannula on the introducer so that it can provide illumination and receive image from the patient's body. The distal tip of the endoscope must remain within a certain range or zone of extension beyond the distal tip of the cannula on the introducer for the endoscope to function properly.

There are many types of introducer devices in the prior art where most are plastic disposable devices. More specifically reference is invited to a conventional and presently available single use introducer an embodiment of which is represented in accompanying FIG. 1. As would be apparent from the illustration in the FIG. 1, such a conventional introducer basically has a metallic-polymeric combination with a front metallic end thereof attached to a metallic cannula and a polymeric material based rear end providing for fixed tubular extension which is there to accommodate a very specific variety of endoscope input connection and at a side thereof a long tubular member for operative connection to insufflations means or dilators, and/or biopsy tools. The unit is integral and a single use introducer. Thus, not only there is limitation in the variety of endoscope that can fit into the conventional introducer due to the integral nature of the endoscope port but also the construction of the introducer has been limited to a single application/use since after use the construction or the material involved were not fit for sterilization for subsequent reuse of the same introducer even with the same endoscope or with any other endoscope or sufflation means.

Thus, no prior art introducers are modular in that they can only connect or couple to certain endoscopes, insufflation flow sources, cannulas or dilators, and/or biopsy tools that have the specific type of fitting or connector that is capable of proper connection with or coupling to the fixed fitting on the non-modular introducer. Thus, prior art introducers severely limit the types of devices that can be used with insufflation endoscopic surgery. This aspect of prior art introducers substantially limits the allowable procedures that can be successfully accomplished with insufflation endoscopic surgery because only certain devices can be connected to the introducer. Also, prior art disposable introducers are by definition single use and cannot be sterilized or autoclaved and thus cannot be used over and over again on different patients. This combined with the fact that prior art disposable introducers are relatively costly in price results in an unnecessary increase in the cost to perform laparoscopic or endoscopic surgery.

It is thus the basic object of the present invention to provide for a modular introducer construction with an introducer hub that would be interchangeable with different types of fittings to accommodate proper connection to and/or coupling with virtually any type of fitting on any endoscope, insufflation flow source, cannula, or dilator.

Another object of the present invention it to provide for a modular introducer with biopsy port in addition to the endoscope and insufflations ports and provide for a biopsy port that would also be interchangeable with different types of fittings to accommodate proper connection to and/or coupling with virtually any type of fitting on any biopsy device or other endoscopic medical device to allow for the use of new medical devices with insufflation endoscopic surgery and also to yield increased precision and control of tissue sample collection with a biopsy device or other endoscopic medical device.

In addition, all components in this invention of a modular introducer which would be repeatedly sterilized by autoclave and thus the introducer of this invention is reusable and capable of much more economical repeated safe use with multiple patients.

### BRIEF SUMMARY OF THE INVENTION

Thus, according to the basic aspect of the present invention there is provided a modular sterilizable and reusable introducer system comprising:

a sterilizable hub comprising a container or vessel body defining an internal hollow passage and having at least two ports comprising an endoscope port and an insufflations port, each said ports communicating with said internal hollow passage of said hub;

said endoscope port comprising a sterilizable endoscope fitting attachment means a sterilizable endoscope fitting for operative connection at one end to said endoscope port through said sterilizable endoscope fitting attachment means in a leak proof manner and at its other end to a cooperative releasable connectable endoscope means in a leak proof manner;

said insufflation port comprising a sterilizable insufflation fitting attachment means a sterilizable insufflation fitting for operative connection at one end to said insufflation port through said sterilizable insufflation fitting attachment means in a leak proof manner and at its other end to a cooperative releasable connectable insufflations means in a leak proof manner;

a sterilizable cannula cooperatively communicating in leak proof manner with said internal hollow passage of said hub.

According to another aspect of the invention there is provided a modular sterilizable and reusable introducer system as above comprising:

a sterilizable hub comprising a container or vessel body defining an internal hollow passage and having at least two ports comprising an endoscope port and an insufflations port and having a cannula entry opening, each said ports and said cannula entry opening communicating with said internal hollow passage of said hub;

said endoscope port comprising a sterilizable endoscope fitting attachment means a sterilizable endoscope fitting for operative connection at one end to said endoscope port through said sterilizable endoscope fitting attachment means in a leak proof manner and at its other end to a cooperative releasable connectable endoscope means in a leak proof manner;

said insufflation port comprising a sterilizable insufflation fitting attachment means a sterilizable insufflation fitting for operative connection at one end to said insufflation port through said sterilizable insufflation fitting attachment means in a leak proof manner and at its other end to a cooperative releasable connectable insufflation means in a leak proof manner;

said cannula entry opening provided with a sterilizable cannula fitting attachment means, a serializable cannula fitting for operative connection at one end to said cannula entry opening through said serializable cannula fitting attachment means in a leak proof manner and at its other end to a cooperative releasable connectable cannula means in a leak proof manner.

According to yet another aspect of the invention there is provided a modular sterilizable and reusable introducer system as above comprising:

a sterilizable hub comprising a container or vessel body defining an internal hollow passage and having at least two ports comprising an endoscope port and an insufflations port and having a cannula entry opening each said port and said cannula entry opening communicating with said internal hollow passage of said hub;

said endoscope port comprising a sterilizable endoscope fitting attachment means a sterilisable endoscope fitting for operative connection at one end to said endoscope port through said sterilizable endoscope fitting attachment means in a leak proof manner and at its other end to a cooperative endoscope means in a leak proof manner;

said insufflation port comprising a sterilizable insufflation fitting attachment means a sterilizable insufflation fitting for operative connection at one end to said insufflation port through said sterilizable insufflation fitting attachment means in a leak proof manner and at its other end to a cooperative insufflation means in a leak proof manner;

said cannula entry opening comprising a serializable cannula fitting attachment means a sterilisable cannula fitting for operative connection at one end to said cannula entry opening through said serializable cannula fitting attachment means in a leak proof manner and at its other end to a cooperative cannula means in a leak proof manner.

This present invention provides a modular sterilizable and reusable introducer system as above wherein said sterilizable hub comprises an oblong shaped container or vessel body with internal hollow passage and has a proximal end which during use if adjacent to surgeon and a distal end opposite to said proximal end which in use is adjacent to the patient and said endoscope port is located at the said proximal end and said cannula entry opening is located at the said distal end of the hub with said insufflations port located at a side of the hub.

According to yet further aspect of the present invention there is provided a modular sterilizable and reusable introducer system as above wherein said hub is capable of containing pressurized flow of gas and/or liquid there through.

According to yet further aspect of the present invention there is provided a modular sterilizable and reusable introducer system as above wherein said endoscope port with endoscope fitting attachment means and said endoscope fitting are capable of coupling and decoupling to numerous types of endoscopes, said cannula entry opening with cannula fitting attachment means and said cannula fitting are capable of coupling and decoupling to numerous types of cannulas, and said insufflations port with insufflations fitting attachment means and said insufflations fitting are capable of coupling and decoupling to numerous types of insufflations means.

According to yet another aspect of the invention there is provided a modular sterilizable and reusable introducer system as above wherein said hub further comprises a biopsy port on a side for insertion of biopsy tool or other endoscope devices.

According to yet another aspect of the present invention there is provided a modular sterilizable and reusable introducer system as above wherein said biopsy port is located directly opposite to the insufflations port and preferably extends from the hub side on a cylindrical shaped biopsy port body such that the longitudinal axes of the hub and the port body form a V shape to ensure that biopsy tool or other endoscopic devices can slide easily with minimal blockage into and out of cannula along with endoscope also inside cannula.

According to another aspect of the present invention there is provided a modular sterilizable and reusable introducer system as above with said biopsy port comprising a sterilizable biopsy or other device fitting attachment means a sterilizable biopsy or other device fitting for operative connection at one end to said biopsy port through said sterilizable biopsy or other device fitting attachment means in a leak proof manner and at its other end to a cooperative biopsy means or other device in a leak proof manner.

According to yet another aspect of the present invention there is provided a modular sterilizable and reusable introducer system as above wherein endoscope fitting, insufflations fitting, cannula fitting, and biopsy fitting is a hydraulic or pneumatic fitting that mates with or couples to hydraulic or pneumatic fittings of endoscope, insufflation, cannula and biopsy/devices.

According to yet another aspect of the present invention there is provided a modular sterilizable and reusable introducer system as above wherein each said endoscope fitting, insufflations fitting, cannula fitting, and biopsy fitting comprise a rigid oblong hollow vessel with a connector end and an attachment end, the connector end adapted to mate with or couple to other hydraulic or pneumatic fittings/devices with corresponding connector ends to yield a leak proof connection between the two fittings and said attachment end connects to said endoscope fitting attachment means, insufflations fitting attachment means, cannula fitting attachment means and biopsy fitting attachment means respectively involving any type of leak proof fitting.

According to another aspect of the present invention there is provided a modular sterilizable and reusable introducer system as above wherein each said endoscope attachment means, said insufflations attachment means, said cannula attachment means and said biopsy attachment means are selected from any cooperative leak proof attachment including selected from threaded male-female attachment, socket attachment, compression ring attachment, luer type taper fitting.

According to another aspect of the present invention there is provided a modular sterilizable and reusable introducer system as above wherein said cannula comprise length of about 2 to 10 centimeters, outer diameter of about 0.3 to 3 millimeters, and wall thickness of about 0.03 to 0.10 millimeter and is obtained as semi-rigid cannula for a small degree of flexibility required to insert and navigate the cannula within the patient preferably the cannula can flex about 1 to 4 millimeters from end to end for this purpose.

According to yet another aspect of the present invention there is provided a modular sterilizable and reusable introducer system as above comprising said cannula is permanently attached or fixed to the hub wherein a proximal end of cannula is positioned around said cannula entry opening and permanently attached to cannula entry opening such as to contain without leaking the pressurized flow of gas or liquid from the interior of hub to the interior of cannula.

According to yet further aspect of the present invention there is provided a modular sterilizable and reusable introducer system as above wherein said leak proof operative connection between the modular components of the hub includes accomplishing the connection involving selectively welded, glued, solvent welded joints.

According to yet further aspect of the present invention there is provided a modular sterilizable and reusable introducer system as above comprising a cannula cover to cover and protect cannula during the autoclave sterilization process to help insure that cannula remains straight and does not bend or warp as a result of the high temperatures reached during the autoclave sterilization process.

According to yet further aspect of the present invention there is provided a modular sterilizable and reusable introducer system as above wherein said cannula cover comprises a retaining cylinder member that is a rigid cylindrical member with open ends, said retaining cylinder having an inner diameter that is sized to make a slip fit over the outside diameter of cannula, the retaining cylinder having a length that is at least as long as that of cannula, a wall thickness that is greater than that of cannula and capable of undergoing sterilization by autoclave without incurring deterioration, degradation, or failure.

According to yet another aspect of the present invention there is provided a method of assembling and disassembling for use and reuse repeated times the modular sterilizable and reusable introducer system as above comprising:

providing said stabilizable hub comprising a container or vessel body defining an internal hollow passage and having at least two ports comprising an endoscope port and an insufflations port and having a cannula entry opening each said ports and a cannulas entry opening communicating with said internal hollow passage of said hub;

operatively connecting to said hub having permanently secured or releasable connected cannula or disconnecting after releasable connection selectively anyone or more of the following modular components involving:

operatively connecting said serializable endoscope fitting at one end to said endoscope port through said sterilizable endoscope fitting attachment means in a leak proof manner and at its other end to a cooperative releasable connectable endoscope means in a leak proof manner;

operatively connecting said sterilizable insufflation fitting at one end to said insufflation port through said sterilizable insufflation fitting attachment means in a leak proof manner and at its other end to a cooperative releasable connectable insufflations means in a leak proof manner;

and operatively connecting said sterilisable biopsy or other device fitting at one end to said biopsy port through said sterilizable biopsy or other device fitting attachment means in a leak proof manner and at its other end to a cooperative biopsy means or other device in a leak proof manner; such as to depending upon the use selectively connect or disconnect to and from said hub depending upon the stage of application of the relevant devices selected from endoscope, insufflations, biopsy or other such releasable connectable device.

According to another aspect there is provided a method as above wherein in case of replaceable cannula use operatively connecting said sterilisable cannula fitting at one end to said cannula entry opening through said sterilizable cannula fitting attachment means in a leak proof manner and at its other end to a cooperative cannula means in a leak proof manner.

The details of the invention, its objects and advantages are explained hereunder in greater detail in relation to non-limiting exemplary illustrations as per the following accompanying figures.

### BRIEF DESCRIPTION OF THE DRAWINGS

FIG. 1: is a front elevation view of a conventional presently available one time use introducer with provision for insufflations;

FIG. 2A is a side elevation view of modular autoclavable introducer with permanent cannula attachment with cannula cover installed.

FIG. 2B is a perspective view from the distal end of modular autoclavable introducer with permanent cannula attachment with cannula cover installed.

FIG. 3 is a side elevation view of cannula cover.

FIG. 4 is a perspective view from the distal end of cannula cover.

FIG. 5 is a side elevation view of modular autoclavable introducer with permanent cannula attachment.

FIG. 6 is a perspective view from the distal end of modular autoclavable introducer with permanent cannula attachment.

FIG. 7A is an exploded view of FIG. 5 with female Luer-type endoscope fitting, female Luer-type insufflation fitting, threaded endoscope fitting attachment means, and threaded insufflation fitting attachment means.

FIG. 7B is an exploded view of FIG. 5 with male Luer-type endoscope fitting, male Luer-type insufflation fitting, threaded endoscope fitting attachment means, and threaded insufflation fitting attachment means.

FIG. 8A is an exploded view of FIG. 5 with female Luer-type endoscope fitting, female Luer-type insufflation fitting, socket endoscope fitting attachment means, and socket insufflation fitting attachment means.

FIG. 8B is an exploded view of FIG. 5 with male Luer-type endoscope fitting, male Luer-type insufflation fitting, socket endoscope fitting attachment means, and socket insufflation fitting attachment means.

FIG. 9 is a side elevation view of hub 20 with reversible cannula attachment and a biopsy port.

FIG. 10 is a perspective view of hub 20 with reversible cannula attachment and a biopsy port.

FIG. 11 is a side elevation view of cannula with female Luer-type hub fitting.

FIG. 12 is a perspective view of cannula with male Luer-type hub fitting.

FIG. 13 is a side elevation view of modular autoclavable introducer with reversible cannula attachment and a biopsy port.

FIG. 14 is a perspective view from the distal end of modular autoclavable introducer with reversible cannula attachment and a biopsy port.

FIG. 15A is an exploded view of FIG. 13 with female Luer-type endoscope fitting, female Luer-type insufflation fitting, female Luer-type biopsy fitting, male Luer-type cannula fitting, threaded endoscope fitting attachment means, threaded insufflation fitting attachment means, threaded biopsy fitting attachment means, and threaded cannula fitting attachment means.

FIG. 15B is an exploded view of FIG. 13 with male Luer-type endoscope fitting, male Luer-type insufflation fitting, male Luer-type biopsy fitting, female Luer-type cannula fitting, threaded endoscope fitting attachment means, threaded insufflation fitting attachment means, threaded biopsy fitting attachment means, and threaded cannula fitting attachment means.

FIG. 16A is an exploded view of FIG. 13 with female Luer-type endoscope fitting, female Luer-type insufflation fitting, female Luer-type biopsy fitting, male Luer-type cannula fitting, socket endoscope fitting attachment means, socket insufflation fitting attachment means, socket biopsy fitting attachment means, and socket cannula fitting attachment means.

FIG. 16B is an exploded view of FIG. 13 with male Luer-type endoscope fitting, male Luer-type insufflation fitting, male Luer-type biopsy fitting, female Luer-type cannula fitting, socket endoscope fitting attachment means, socket insufflation fitting attachment means, socket biopsy fitting attachment means, and socket cannula fitting attachment means.

FIG. 17 is a cross-sectional view of modular autoclavable introducer with permanent cannula attachment along with an endoscope inserted therein during endoscopic surgery with the flow of insufflation gas or liquid.

FIG. 18 is a cross-sectional view of modular autoclavable introducer with permanent cannula attachment and a biopsy port along with an endoscope and a biopsy device inserted therein during endoscopic surgery with the flow of insufflation gas or liquid.

### DEFINITION LIST

| Term | Definition |
|---|---|
| 10 | Modular Autoclavable Introducer |
| 20 | Hub |
| 21 | Endoscope Port |
| 22 | Endoscope Fitting |
| 23 | Endoscope Fitting Attachment Means |
| 24 | Insufflation Port |
| 25 | Insufflation Fitting |
| 26 | Insufflation Attachment Means |
| 27 | Cannula Port |
| 28 | Cannula Fitting |
| 29 | Cannula Fitting Attachment Means |
| 30 | Cannula |
| 32 | Distal End of Cannula |
| 34 | Proximal End of Cannula |
| 36 | Hub Fitting |
| 40 | Biopsy Port |
| 42 | Biopsy Port Body |
| 44 | Biopsy Fitting |
| 46 | Biopsy Fitting Attachment Means |
| 50 | Cannula Cover |
| 60 | Endoscope |
| 70 | Insufflation Gas or Liquid |
| 80 | Biopsy Tool or other Endoscopic Device |
| 100 | Patient Tissue |

### DETAILED DESCRIPTION OF THE INVENTION

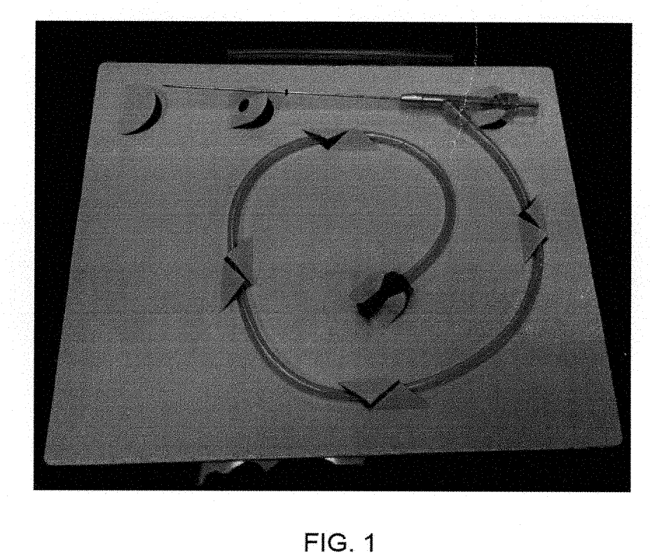

The reference to FIG. 1 shows an embodiment of a conventional single use introducer in accordance with the present invention. As would be clearly apparent from the FIG. 1 such a conventional introducer basically has a metallic-polymeric combination with a front metallic end thereof attached to a metallic cannula and a polymeric material based rear end providing for fixed tubular extension which is there to accommodate a very specific variety of endoscope input connection and at a side thereof a long tubular member for operative connection to insufflations means or dilators, and/or biopsy tools. The unit is integral and a single use introducer. Thus, not only there is limitation in the variety of endoscope that can fit into the conventional introducer due to the integral nature of the endoscope port but also the construction of the introducer has been limited to a single application/use since after use the construction or the material involved were not fit for sterilization for subsequent reuse of the same introducer even with the same endoscope or with any other endoscope or sufflation means.

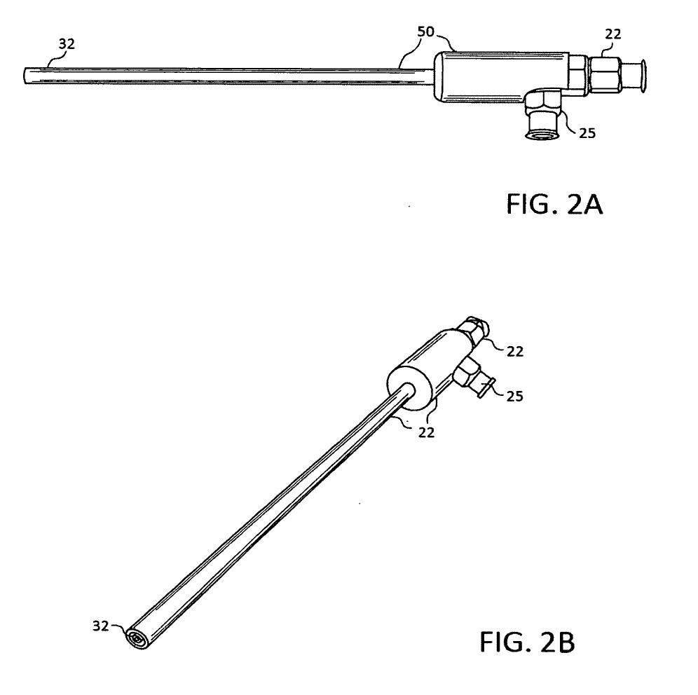

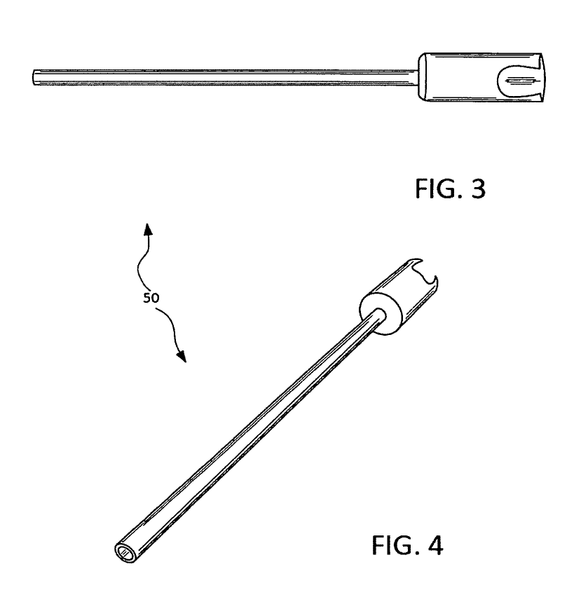

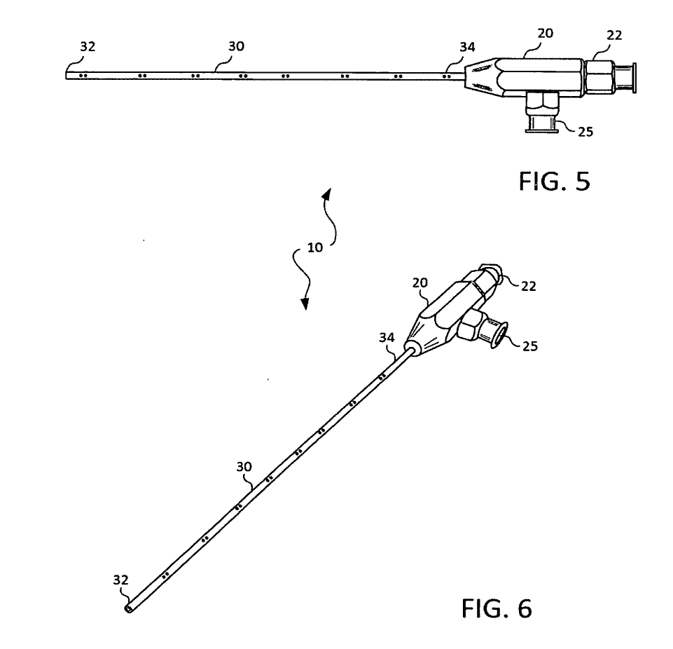

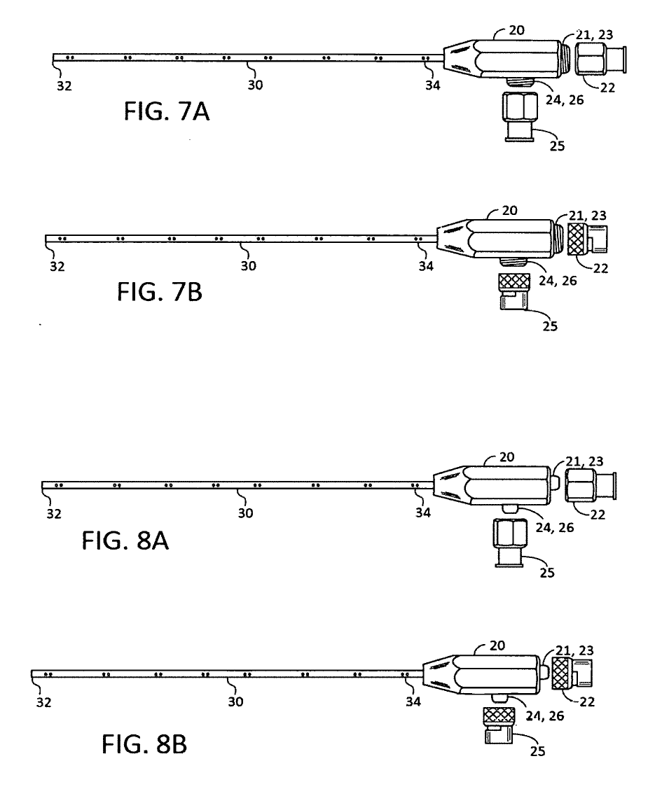

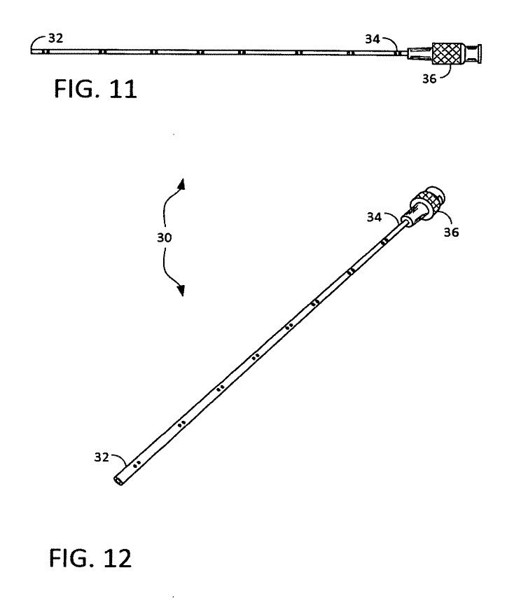

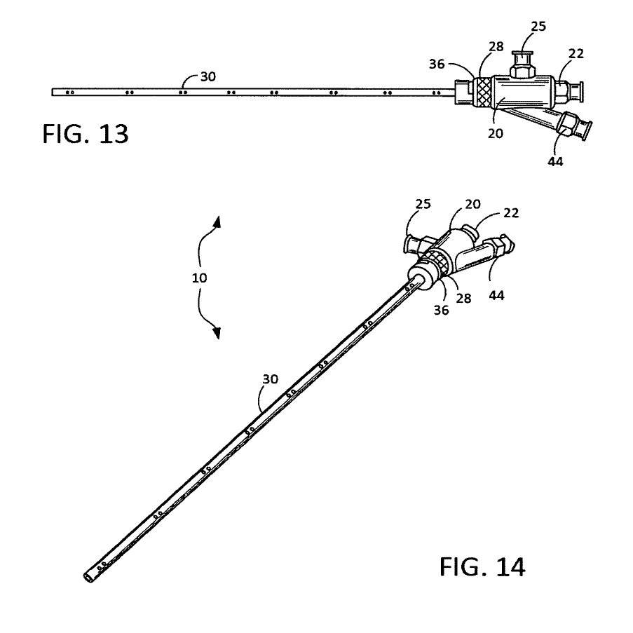

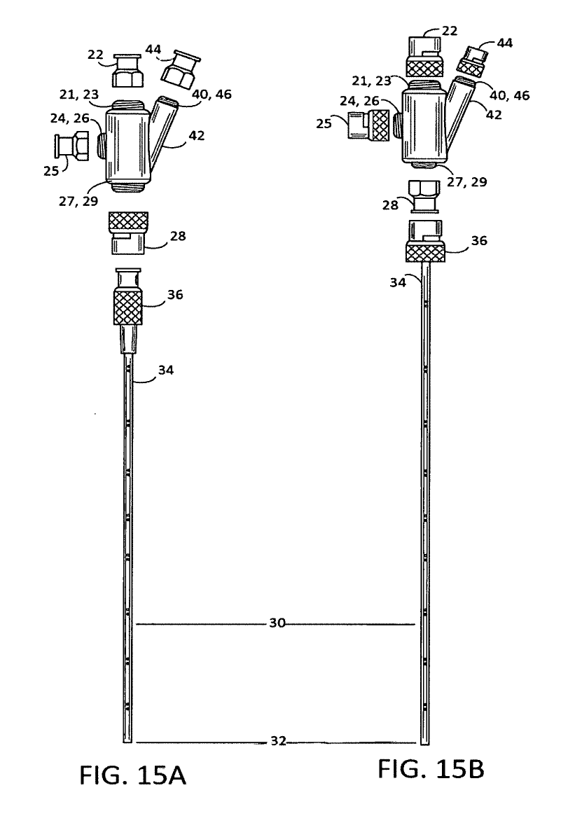

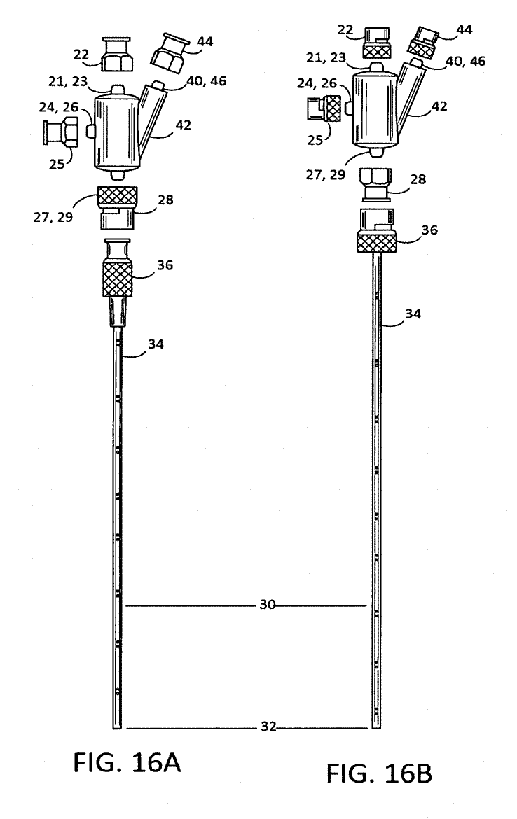

The illustrations of the modular autoclavable and reusable introducer in accordance with the present advancement as illustrated in relation to accompanying FIGS. 2A to 18.

The modular autoclavable introducer 10 of the invention comprises a hub 20 coupled to a cannula 30. Hub 20 is an oblong rigid hollow container or vessel with at least three openings or ports therein, a proximal end, and a distal end. In use, the proximal end is position adjacent to the surgeon and the distal end is held adjacent to the patient to perform surgery. At least three openings in hub 20 comprise: an endoscope port 21 located on the proximal end of hub 20, an insufflation port 24 located on the side of hub 20, and a cannula port 27 located on the distal end of hub 20. Hub 20 is capable of containing the pressurized flow of gas or liquid there through. With endoscope port 21 sealed or plugged to prevent the flow of liquid or gas there through hub 20 is capable of containing the pressurized flow of gas or liquid that enters through insufflation port 24, travels along the longitudinal axis of hub 20, and exits through cannula port 27, without leaking through the oblong rigid hollow container or vessel.

Hub 20 must be capable of undergoing sterilization by autoclave without incurring deterioration or degradation. Hub 20 further comprises an endoscope fitting 22 reversibly coupled to endoscope port 21 by an endoscope fitting attachment means 23. Endoscope fitting 22 is a hydraulic or pneumatic fitting those mates with or couples to other hydraulic or pneumatic fittings. Endoscope fitting 22 is a rigid oblong hollow vessel with a connector end and an attachment end. Connector end has a certain shape that mates with or couples to other hydraulic or pneumatic fittings with other certain shaped connector ends to yield a leak proof connection between the two fittings. The attachment end connects to endoscope fitting attachment means 23. Endoscope fitting attachment means 23 is a means to couple endoscope fitting 22 to hub 20, positioned around endoscope port 21, and in such a way so as to contain without leaking the pressurized flow of gas or liquid from hub 20 to endoscope fitting 22.

Endoscope fitting attachment means 23 can be accomplished by threaded attachment where female threads are inscribed onto the inner diameter of the attachment end of endoscope fitting 22 that are sized to mate with male threads inscribed onto the exterior of hub 20, to provide leak-proof reversible threaded attachment between these members. Endoscope fitting attachment means 23 can be accomplished by threaded attachment where male threads are inscribed onto the outer diameter of the attachment end of endoscope fitting 22 that are sized to mate with female threads inscribed onto the interior of hub 20, to provide leak-proof reversible threaded attachment between these members.

Endoscope fitting attachment means 23 can be accomplished by socket attachment where a female socket is formed in the inner diameter of the attachment end of endoscope fitting 22 that is sized to form a slip-fit with a male socket formed on the exterior of hub 20, where female and male sockets are nested together and welded, glued, soldered, brazed, or solvent welded in place to provide leak-proof permanent socket attachment between these members. Endoscope fitting attachment means 23 can be accomplished by socket attachment where a male socket is formed in the outer diameter of the attachment end of endoscope fitting 22 that is sized to form a slip-fit with a female socket formed on the interior of hub 20, where female and male sockets are nested together and welded, glued, soldered, brazed, or solvent welded in place to provide leak-proof permanent socket attachment between these members.

Endoscope fitting attachment means 23 can be accomplished by compression ring attachment where a resilient compression ring is inserted between a female endoscope fitting 22 and a male hub 20 or a male endoscope fitting 22 and a female hub 20 and a nut or clamp is used to compress the compression ring between these members to form a leak-proof reversible compression attachment between these members.

Endoscope fitting 22 and endoscope fitting attachment means 23 must be capable of undergoing sterilization by autoclave. In best mode, endoscope fitting 22 is made of surgical stainless steel. In best mode, endoscope fitting 22 is a Luer type taper fitting that is capable of mating with other Luer type taper fittings. Luer type taper fittings reversibly couple together by a male convex hollow taper end from one Luer type taper fitting being inserted and pressed against a female concave hollow taper end from another Luer type taper fitting to provide a leak-proof reversible attachment between these two Luer type taper fittings. Luer type fittings have connection ends that are designated as either male or female based on these criteria. Typically, a helical notch on a collar on the male end receives a lip on the female end where the helical notch and/or the lip is rotated to compress female taper against male taper and vice versa to properly couple the two Luer type taper fittings together.

Hub 20 further comprises an insufflation fitting 25 reversibly coupled to insufflation port 24 by an insufflation fitting attachment means 26. Insufflation fitting 25 is a hydraulic or pneumatic fitting those mates with or couples to other hydraulic or pneumatic fittings. Insufflation fitting 25 is a rigid oblong hollow vessel with a connector end and an attachment end. Connector end has a certain shape that mates with or couples to other hydraulic or pneumatic fittings with other certain shaped connector ends to yield a leak proof connection between the two fittings. The attachment end connects to insufflation fitting attachment means 26. Any known or unknown type of fitting may be used here. Insufflation fitting attachment means 26 is a means to couple insufflation fitting 25 to hub 20, positioned around insufflation port 24, and in such a way so as to contain without leaking the pressurized flow of gas or liquid from hub 20 to insufflation fitting 25.

Insufflation fitting attachment means 26 can be accomplished by threaded attachment where female threads are inscribed onto the inner diameter of the attachment end of insufflation fitting 25 that are sized to mate with male threads inscribed onto the exterior of hub 20, to provide leak-proof reversible threaded attachment between these members. Insufflation fitting attachment means 26 can be accomplished by threaded attachment where male threads are inscribed onto the outer diameter of the attachment end of insufflation fitting 25 that are sized to mate with female threads inscribed onto the interior of hub 20, to provide leak-proof reversible threaded attachment between these members.

Insufflation fitting attachment means 26 can be accomplished by socket attachment where a female socket is formed in the inner diameter of the attachment end of insufflation fitting 25 that is sized to form a slip-fit with a male socket formed on the exterior of hub 20, where female and male sockets are nested together and welded, glued, soldered, brazed, or solvent welded in place to provide leak-proof permanent socket attachment between these members. Insufflation fitting attachment means 26 can be accomplished by socket attachment where a male socket is formed in the outer diameter of the attachment end of insufflation fitting 25 that is sized to form a slip-fit with a female socket formed on the interior of hub 20, where female and male sockets are nested together and welded, glued, soldered, brazed, or solvent welded in place to provide leak-proof permanent socket attachment between these members.

Insufflation fitting attachment means 26 can be accomplished by compression ring attachment where a resilient compression ring is inserted between a female insufflation fitting 25 and a male hub 20 or a male insufflation fitting 25 and a female hub 20 and a nut or clamp is used to compress the compression ring between these members to form a leak-proof reversible compression attachment between these members.

Insufflation fitting 25 and insufflation fitting attachment means 26 must be capable of undergoing sterilization by autoclave without incurring deterioration, degradation, or failure. In best mode, endoscope fitting 22 is made of surgical stainless steel. In best mode, insufflation fitting 25 is a Luer type taper fitting that is capable of mating with other Luer type taper fittings.

Cannula 30 is a semi-rigid tube or cylindrical-shaped member with open ends. Cannula 30 has a proximal end 34 and a distal end 32. The distal end 32 of cannula 30 is inserted into the patient's body for use with endoscopic surgery as described above. The proximal end 34 of cannula 30 is attached to the distal end of hub 20. Cannula 30 is semi-rigid because a small degree of flexibility is required to insert and navigate the cannula 30 within the patient.

Cannula 30 may flex about 1 to 4 millimeters from end to end for this purpose. However, cannula 30 may not flex more than this amount because a certain amount of rigidity is required to properly insufflate the target area of tissue and navigate the endoscope as described above. Cannula 30 has length of about 2 to 10 centimeters, outer diameter of about 0.3 to 3 millimeters, and wall thickness of about 0.03 to 0.10 millimeters. Cannula 30 must be capable of sterilization by autoclave without incurring deterioration or degradation. In some modes of the invention, the proximal end 34 of cannula 30 is permanently coupled or attached to hub 20. Thus, these members may not be disconnected and there is no cannula fitting 28 or hub fitting 36 in these modes. In permanent cannula attachment modes, the proximal end 34 of cannula 30 is positioned around cannula port 27 and permanently attached to cannula port 27 in such a way so as to contain without leaking the pressurized flow of gas or liquid from the interior of hub 20 to the interior of cannula 30. A female socket is formed in the inner diameter of the distal end of hub 20 that is sized to form a slip-fit with a male socket that is the exterior surface of the proximal end 34 of cannula 30, where female and male sockets are nested together and welded, glued, soldered, brazed, or solvent welded in place to provide leak-proof permanent socket attachment between these members that is capable of undergoing sterilization by autoclave without incurring deterioration or degradation. These modes are useful because the modular aspect or the ability to connect to numerous types of fittings and thereby capable of coupling and decoupling to numerous types of endoscopes, cannulas, insufflation flow sources, biopsy devices, and other medical devices is primarily desired with the endoscope port 21 connection and the insufflation port 24 connection. In many cases, it is preferred that the connection between hub 20 and cannula 30 remain permanent and non-modular. Examples of these modes are depicted in FIGS. 1, 2, 5-8B, 17, and 18.

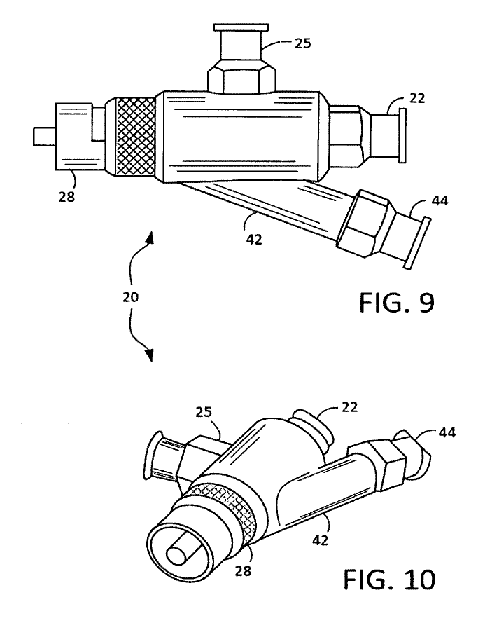

In other modes of the invention, cannula 30 is reversibly coupled or attached to hub 20. With these modes, it is preferred that the modular aspect extend to the cannula port 27 connection along with the endoscope port 21 connection and the insufflation port 24 connection. Examples of these modes are depicted in FIGS. 9-16B.

In reversible cannula attachment modes, the distal end of hub 20 further comprises a cannula fitting 28 reversibly coupled to cannula port 27 by a cannula fitting attachment means 29. Cannula fitting 28 is a hydraulic or pneumatic fitting those mates with or couples to other hydraulic or pneumatic fittings. Cannula fitting 28 is a rigid oblong hollow vessel with a connector end and an attachment end. Connector end has a certain shape that mates with or couples to other hydraulic or pneumatic fittings with other certain shaped connector ends to yield a leak proof connection between the two fittings. The attachment end connects to cannula fitting attachment means 29. Any known or unknown type of fitting may be used here. Cannula fitting attachment means 29 is a means to couple cannula fitting 28 to hub 20, positioned around cannula port 27, and in such a way so as to contain without leaking the pressurized flow of gas or liquid from hub 20 to cannula fitting 28.

Cannula fitting attachment means 29 can be accomplished by threaded attachment where female threads are inscribed onto the inner diameter of the attachment end of cannula fitting 28 that are sized to mate with male threads inscribed onto the exterior of hub 20, to provide leak-proof reversible threaded attachment between these members. Cannula fitting attachment means 29 can be accomplished by threaded attachment where male threads are inscribed onto the outer diameter of the attachment end of cannula fitting 28 that are sized to mate with female threads inscribed onto the interior of hub 20, to provide leak-proof reversible threaded attachment between these members.

Cannula fitting attachment means 29 can be accomplished by socket attachment where a female socket is formed in the inner diameter of the attachment end of cannula fitting 28 that is sized to form a slip-fit with a male socket formed on the exterior of hub 20, where female and male sockets are nested together and welded, glued, soldered, brazed, or solvent welded in place to provide leak-proof permanent socket attachment between these members. Cannula fitting attachment means 29 can be accomplished by socket attachment where a male socket is formed in the outer diameter of the attachment end of cannula fitting 28 that is sized to form a slip-fit with a female socket formed on the interior of hub 20, where female and male sockets are nested together and welded, glued, soldered, brazed, or solvent welded in place to provide leak-proof permanent socket attachment between these members.

Cannula fitting attachment means 29 can be accomplished by compression ring attachment where a resilient compression ring is inserted between a female cannula fitting 28 and a male hub 20 or a male cannula fitting 28 and a female hub 20 and a nut or clamp is used to compress the compression ring between these members to form a leak-proof reversible compression attachment between these members.

Cannula fitting 28 and cannula fitting attachment means 29 must be capable of undergoing sterilization by autoclave without incurring deterioration, degradation, or failure. Cannula fitting 28 is made of a material with a melting temperature greater than 1000 degrees Fahrenheit such as metal, steel, steel alloy, ceramic, composite, or certain plastics. In best mode, cannula fitting 28 is made of surgical stainless steel. In best mode, cannula fitting 28 is a Luer type taper fitting that is capable of mating with other Luer type taper fittings.

In reversible cannula attachment modes, the proximal end 34 of cannula 30 is permanently coupled or attached to a hub fitting 36. Hub fitting 36 is a hydraulic or pneumatic fitting those mates with or couples to other hydraulic or pneumatic fittings. Hub fitting 36 is a rigid oblong hollow vessel with a connector end and an attachment end. Connector end has a certain shape that mates with or couples to other hydraulic or pneumatic fittings with other certain shaped connector ends to yield a leak proof connection between the two fittings. Any known or unknown type of fitting may be used here. A female socket is formed in the inner diameter of the attachment end of hub fitting 36 that is sized to form a slip-fit with a male socket that is the exterior surface of the proximal end 34 of cannula 30, where female and male sockets are nested together and welded, glued, soldered, brazed, or solvent welded in place to provide leak-proof permanent socket attachment between these members that is capable of undergoing sterilization by autoclave without incurring deterioration or degradation.

Hub fitting 36 must be capable of undergoing sterilization by autoclave without incurring deterioration, degradation, or failure. In best mode, hub fitting 36 is made of surgical stainless steel. In best mode, hub fitting 36 is a taper fitting that is capable of mating with other Luer type taper fittings.

Hub 20 may further comprise a biopsy port 40 and a biopsy port body 42. Biopsy port 40 is an additional opening or port in hub 20 to the three openings or ports described above. Biopsy port 40 is positioned on the side of hub 20, on the direct directly opposite side from that where insufflation port 24 is located. Biopsy port 40 is used to insert a biopsy tool or other endoscopic device 80 into the small incision or natural opening that is the operation site along with the endoscope 60 in order to take a biopsy tissue sample from the target area and remove the tissue sample from the small incision or natural opening or to perform another endoscopic medical procedure.

Biopsy port body 42 is a rigid cylindrical member with open ends. Biopsy port body 42 has a distal end and a proximal end. With open ends sealed or plugged to prevent the flow of liquid or gas there through, biopsy port body 42 is capable of containing the pressurized flow of gas or liquid therein without leaking. The distal end 34 of biopsy port body 42 is positioned around biopsy port 40 and permanently attached to biopsy port 40 in such a way so as to contain without leaking the pressurized flow of gas or liquid from the interior of hub 20 to the interior of biopsy port body 42. This attachment being accomplished by welding, gluing, soldering, brazing, or solvent welding to provide leak-proof permanent socket attachment between these members,

that is capable of undergoing sterilization by autoclave without incurring deterioration or degradation. With this attachment, the proximal end of biopsy port body 42 forms an acute angle with the proximal end of hub 20 so that the longitudinal axes of hub 20 and biopsy port body 42 form a V-shape. This design ensures that the biopsy tool or other endoscopic device 80 may slide more easily with minimal blockage into and out of cannula 30 along with endoscope 60 also inside cannula 30.

Biopsy port modes of the invention include a biopsy fitting 44. The proximal end of biopsy port body includes a biopsy fitting attachment means 46. Biopsy fitting 44 reversibly couples to biopsy port body 42 by biopsy fitting attachment means 46. Biopsy fitting 44 is a hydraulic or pneumatic fitting those mates with or couples to other hydraulic or pneumatic fittings. Biopsy fitting 44 is a rigid oblong hollow vessel with a connector end and an attachment end.

Connector end has a certain shape that mates with or couples to other hydraulic or pneumatic fittings with other certain shaped connector ends to yield a leak proof connection between the two fittings. The attachment end connects to biopsy fitting attachment means 46. Any known or unknown type of fitting may be used here. Biopsy fitting attachment means 46 is a means to couple biopsy fitting 44 to biopsy port body 42 in such a way so as to contain without leaking the pressurized flow of gas or liquid from biopsy port body 42 to biopsy fitting 44.

Biopsy fitting attachment means 46 can be accomplished by threaded attachment where female threads are inscribed onto the inner diameter of the attachment end of biopsy fitting 44 that are sized to mate with male threads inscribed onto the exterior of hub 20, to provide leak-proof reversible threaded attachment between these members. Biopsy fitting attachment means 46 can be accomplished by threaded attachment where male threads are inscribed onto the outer diameter of the attachment end of biopsy fitting 44 that are sized to mate with female threads inscribed onto the interior of hub 20, to provide leak-proof reversible threaded attachment between these members.

Biopsy fitting attachment means 46 can be accomplished by socket attachment where a female socket is formed in the inner diameter of the attachment end of biopsy fitting 44 that is sized to form a slip-fit with a male socket formed on the exterior of hub 20, where female and male sockets are nested together and welded, glued, soldered, brazed, or solvent welded in place to provide leak-proof permanent socket attachment between these members. Biopsy fitting attachment means 46 can be accomplished by socket attachment where a male socket is formed in the outer diameter of the attachment end of biopsy fitting 44 that is sized to form a slip-fit with a female socket formed on the interior of hub 20, where female and male sockets are nested together and welded, glued, soldered, brazed, or solvent welded in place to provide leak-proof permanent socket attachment between these members.

Biopsy fitting attachment means 46 can be accomplished by compression ring attachment where a resilient compression ring is inserted between a female biopsy fitting 44 and a male hub 20 or a male biopsy fitting 44 and a female hub 20 and a nut or clamp is used to compress the compression ring between these members to form a leak-proof reversible compression attachment between these members.

Biopsy fitting 44 and biopsy fitting attachment means 46 must be capable of undergoing sterilization by autoclave. In best mode, biopsy fitting 44 is made of surgical stainless steel. In best mode, biopsy fitting 44 is a Luer type taper fitting that is capable of mating with other Luer type taper fittings.

Optionally, modular autoclavable introducer 10 may include a cannula cover 50 to cover and protect cannula 30 during the autoclave sterilization process to help insure that cannula 20 remains straight and does not bend or warp as a result of the high temperatures reached during the autoclave sterilization process. Cannula cover 50 comprises a retaining cylinder member that is a rigid cylindrical member with open ends. Retaining cylinder has an inner diameter that is sized to make a slip fit over the outside diameter of cannula 30. Retaining cylinder has a length that is at least as long as that of cannula 30. Retaining cylinder has a wall thickness that is greater than that of cannula 30. Cannula cover 50 must be capable of undergoing sterilization by autoclave without incurring deterioration, degradation, or failure. In best mode, cannula cover 50 is made of surgical stainless steel. Cannula cover 50 is slid over cannula 30 prior to and during the sterilization process and functions to prevent cannula from bending or warping during sterilization. After the sterilization process is completed and the cannula 30 is cooled, cannula cover 50 is removed to use the cannula 30 as detailed here within.

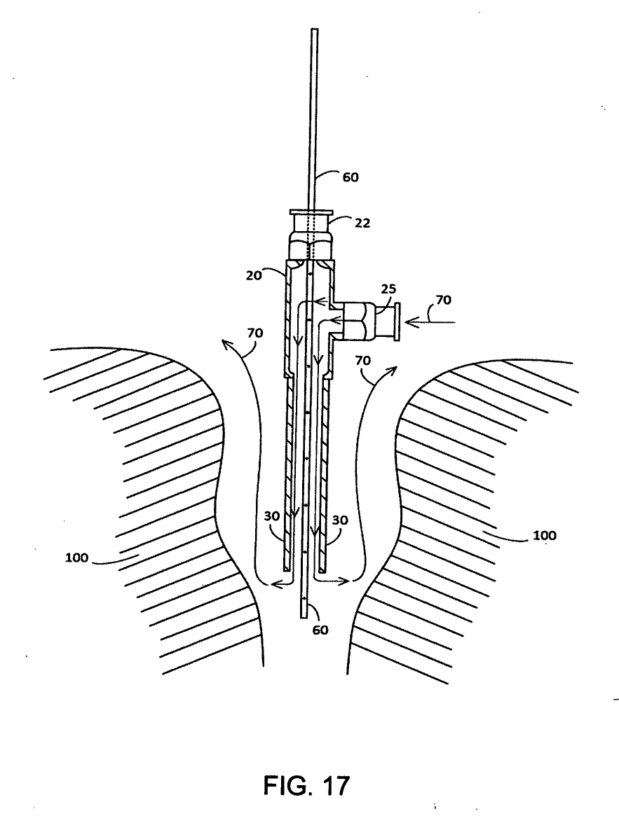

Modular autoclavable introducer 10 can be used to help perform endoscopic surgery with an endoscope 60 as follows. First, the surgeon determines what particular type of endoscope 60 he or she would like to use during the surgery and what particular type of fitting is affixed to the chosen endoscope. Next, the surgeon determines what particular type of insufflation source he or she would like to use during the surgery and what particular type of fitting is affixed to the chosen insufflation source. Then, the particular type of endoscope fitting 22 that will properly couple to the particular fitting on the chosen endoscope is determined where this fitting 22 is then attached to hub 20 with endoscope fitting attachment means 23 as described above. Also, the particular type of insufflation fitting 25 that will properly couple to the particular fitting on the chosen insufflation source is determined where this fitting 25 is then attached to hub 20 with insufflation fitting attachment means 26 as described above. Next, the chosen endoscope 60 is coupled to modular autoclavable introducer 10 by threading the distal end of endoscope 60 through endoscope fitting 22, hub 20, cannula 30, and then out through the distal end 32 of cannula 30 and coupling the fitting (not depicted) on endoscope 60 to the endoscope fitting 22 on hub 20. With this connection completed, the flow of insufflation gas or liquid 70 cannot flow back through endoscope port 21. Next, the insufflation flow source is coupled to modular autoclavable introducer 10 by coupling the fitting (not depicted) on the insufflation flow source to insufflation fitting 25 on hub 20. With this connection completed, the insufflation flow source is then turned on to start the flow insufflation gas or liquid 70 from insufflation flow source through insufflation port 24, hub 20, cannula 30, and then out through the distal end 32 of cannula 30. Next, the modular autoclavable introducer 10 and endoscope 60 are inserted into the small incision or natural opening so that insufflation gas or liquid 70 expands patient tissue 100 as depicted in FIG. 17 to perform endoscopic surgery. Typically, the small incision or natural opening that is the operation site is dilated using a dilating device in preparation for the initial insertion of modular autoclavable introducer 10 and endoscope 60 and prior to the insertion of modular autoclavable introducer 10 and endoscope 60. Endoscopic surgery is then conducted by moving modular autoclavable introducer 10 and endoscope 60 within the small incision or natural opening.

Afterward endoscopic surgery is completed; endoscope 60 and modular autoclavable introducer 10 are removed from the small incision or natural opening operation. The insufflation flow source is then turned off and decoupled from hub insufflation fitting 25 and removed from hub 20. The endoscope 60 is then decoupled from endoscope fitting 22 and removed from hub 20. Modular autoclavable introducer 10 is then sterilized by autoclave after the surgery for repeated surgical use with many patients.

Modular autoclavable introducer 10 can be used to help perform endoscopic surgery with an endoscope 60 as follows. First, the surgeon determines what particular type of endoscope 60 he or she would like to use during the surgery and what particular type of fitting is affixed to the chosen endoscope. Next, the surgeon determines what particular type of insufflation source he or she would like to use during the surgery and what particular type of fitting is affixed to the chosen insufflation source. Next, the surgeon determines what particular type of cannula or dilator 30 he or she would like to use during the surgery and what particular type of fitting is affixed to the chosen cannula or dilator. Next, the surgeon determines what particular type of biopsy tool or other endoscopic medical device 80 he or she would like to use during the surgery and what particular type of fitting is affixed to the chosen biopsy tool. Then, the particular type of endoscope fitting 22 that will properly couple to the particular fitting on the chosen endoscope is determined where this fitting 22 is then attached to hub 20 with endoscope fitting attachment means 23 as described above. Also, the particular type of insufflation fitting 25 that will properly couple to the particular fitting on the chosen insufflation source is determined where this fitting 25 is then attached to hub 20 with insufflation fitting attachment means 26 as described above.

Also, the particular type of cannula fitting 28 that will properly couple to the particular fitting on the chosen cannula or dilator is determined where this fitting 28 is then attached to hub 20 with cannula fitting attachment means 29 as described above. Also, the particular type of biopsy fitting 44 that will properly couple to the particular fitting on the chosen biopsy tool or other endoscopic device 80 is determined where this fitting 44 is then attached to biopsy port body 42 with biopsy fitting attachment means 46 as described above. Next, the chosen endoscope 60 is coupled to modular autoclavable introducer 10 by threading the distal end of endoscope 60 through endoscope fitting 22, hub 20, cannula 30, and then out through the distal end 32 of cannula 30 and coupling the fitting (not depicted) on endoscope 60 to the endoscope fitting 22 on hub 20. With this connection completed, the flow of insufflation gas or liquid 70 cannot flow back through endoscope port 21.

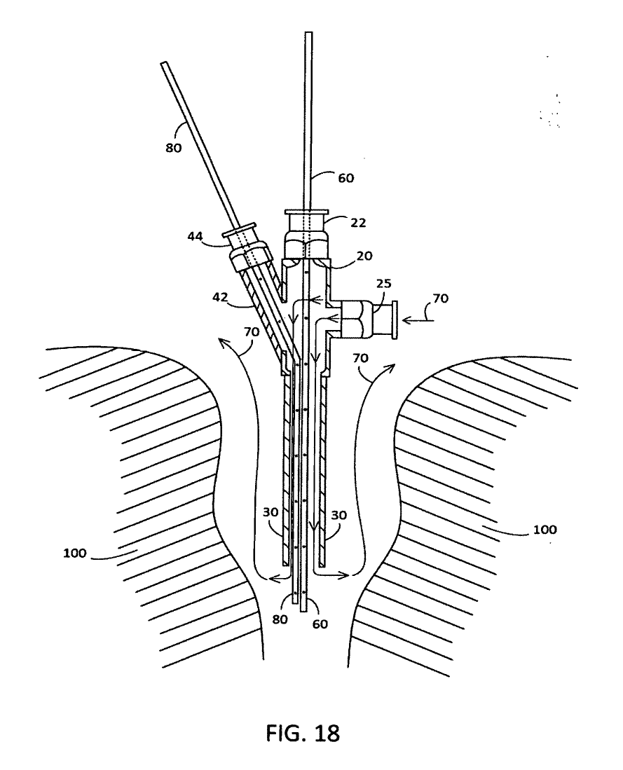

Next, the chosen insufflation flow source is coupled to modular autoclavable introducer 10 by coupling the fitting (not depicted) on the insufflation flow source to insufflation fitting 25 on hub 20. Next, the chosen cannula or dilator 30 is coupled to modular autoclavable introducer 10 by coupling hub fitting 36 on cannula 30 to cannula fitting 28 on hub 20. Next, a cap or plug is installed over biopsy fitting 44 to prevent the flow of insufflation gas or liquid 70 back through biopsy port 40. Next, the insufflation flow source is then turned on to start the flow insufflation gas or liquid 70 from insufflation flow source through insufflation port 24, hub 20, cannula 30, and then out through the distal end 32 of cannula 30. Next, the modular autoclavable introducer 10 and endoscope 60 are inserted into the small incision or natural opening so that insufflation gas or liquid 70 expands patient tissue 100 as depicted in FIG. 18 to perform endoscopic surgery. Typically, the small incision or natural opening that is the operation site is dilated using a dilating device in preparation for the initial insertion of modular autoclavable introducer 10 and endoscope 60 and prior to the insertion of modular autoclavable introducer 10 and endoscope 60. The surgeon then navigates modular autoclavable introducer 10 and endoscope 60 to a target tissue area. Next, the chosen biopsy tool or other endoscopic device 80 is coupled to modular autoclavable introducer 10 by threading the distal end of biopsy tool or other endoscopic device 80 through biopsy fitting 44, biopsy port body 42, hub 20, cannula 30, and then out through the distal end 32 of cannula 30 and coupling the fitting (not depicted) on biopsy tool or other endoscopic device 80 to the biopsy fitting 44 on biopsy port body 42 as depicted in FIG. 18. Biopsy tool or other endoscopic device 80 is then used to collect a biopsy tissue sample or perform another endoscopic procedure. After a biopsy tissue sample has been collected or other endoscopic procedure has been completed, biopsy tool or other endoscopic device 80, endoscope 60, and modular autoclavable introducer 10 are removed from the small incision or natural opening operation. The insufflation flow source is then turned off and decoupled from hub insufflation fitting 25 and removed from hub 20. The biopsy tool or other endoscopic device 80 is then decoupled from biopsy fitting 22 and removed from hub 20.

The endoscope 60 is then decoupled from endoscope fitting 22 and removed from hub 20. The cannula or dilator 30 is then decoupled from cannula fitting 28 and removed from hub 20. All components of modular autoclavable introducer 10 are then sterilized by autoclave after the surgery for repeated surgical use with many patients.

It is thus possible by way of the present invention to provide for an endoscopic or laparoscopic introducer and specifically to an autoclavable modular introducer which is modular in that it is capable of attachment to numerous types of fittings and thereby capable of coupling and decoupling to numerous types of endoscopes, cannulas, insufflation flow sources, biopsy devices, and other medical devices and importantly the introducer of the present advancement having modular construction is also autoclavable in that it is capable of safely and efficiently sterilized by autoclave without incurring deterioration or degradation.

1. A modular sterilizable and reusable introducer system comprising:
a sterilizable hub comprising a container or vessel body defining an internal hollow passage and having at least two ports comprising an endoscope port and an insufflations port, each said ports communicating with said internal hollow passage of said hub; said endoscope port comprising a sterilizable endoscope fitting attachment means a sterilizable endoscope fitting for operative connection at one end to said endoscope port through said sterilizable endoscope fitting attachment means in a leak proof manner and at its other end to a cooperative releasable connectable endoscope means in a leak proof manner; said insufflation port comprising a sterilizable insufflation fitting attachment means a sterilizable insufflation fitting for operative connection at one end to said insufflation port through said sterilizable insufflation fitting attachment means in a leak proof manner and at its other end to a cooperative releasable connectable insufflations means in a leak proof manner; a sterilizable cannula cooperatively communicating in a leak proof manner with said internal hollow passage of said hub.

2. A modular sterilizable and reusable introducer system is claimed comprising:
a sterilizable hub comprising a container or vessel body defining an internal hollow passage and having at least two ports comprising an endoscope port and an insufflations port, and having a cannula entry opening each said port and said cannula entry opening communicating with said internal hollow passage of said hub; said endoscope port comprising a sterilizable endoscope fitting attachment means a sterilizable endoscope fitting for operative connection at one end to said endoscope port through said sterilizable endoscope fitting attachment means in a leak proof manner and at its other end to a cooperative releasable connectable endoscope means in a leak proof manner; said insufflation port comprising a sterilizable insufflation fitting attachment means:
a sterilisable insufflation fitting for operative connection at one end to said insufflation port through said sterilizable insufflation fitting attachment means in a leak proof manner and at its other end to a cooperative releasable connectable insufflations means in a leak proof manner;
said cannula entry opening provided with a sterilizable cannula fitting attachment means a sterilisable cannula fitting for operative connection at one end to said cannula entry opening through said sterilizable cannula fitting attachment means in a leak proof manner and at its other end to a cooperative releasable connectable cannula means in a leak proof manner.

3. A modular sterilizable and reusable introducer system is claimed:
a sterilizable hub comprising a container or vessel body defining an internal hollow passage and having at least two ports comprising an endoscope port and an insufflations port, and having a cannula entry opening, each said ports and said cannula entry opening communicating with said internal hollow passage of said hub; said endoscope port comprising a sterilizable endoscope fitting attachment means a sterilizable endoscope fitting for operative connection at one end to said endoscope port through said sterilizable endoscope fitting attachment means in a leak proof manner and at its other end to a cooperative endoscope means in a leak proof manner; said insufflation port comprising a sterilizable insufflation fitting attachment means:
a sterilisable insufflation fitting for operative connection at one end to said insufflation port through said sterilizable insufflation fitting attachment means in a leak proof manner and at its other end to a cooperative insufflations means in a leak proof manner; said cannula entry opening comprising a sterilizable cannula fitting attachment means:
a sterilisable cannula fitting for operative connection at one end to said cannula entry opening through said sterilizable cannula fitting attachment means in a leak proof manner and at its other end to a cooperative cannula means in a leak proof manner.

4. A modular sterilizable and reusable introducer system is claimed wherein said sterilizable hub comprises an oblong shaped container or vessel body with internal hollow passage and has a proximal end which during use if adjacent to surgeon and a distal end opposite to said proximal end which in use is adjacent to the patient and said endoscope port is located at the said proximal end and said cannula entry opening is located at the said distal end of the hub with said insufflations port located at a side of the hub.

5. A modular sterilizable and reusable introducer system as claimed: wherein said endoscope port with endoscope fitting attachment means and said endoscope fitting are capable of coupling and decoupling to numerous types of endoscopes, said cannula entry opening with cannula fitting attachment means and said cannula fitting are capable of coupling and decoupling to numerous types of cannulas, and said insufflations port with insufflations fitting attachment means and said insufflations fitting are capable of coupling and decoupling to numerous types of insufflations means.

6. A modular sterilizable and reusable introducer system as claimed: wherein said hub further comprises a biopsy port on a side for insertion of biopsy tool or other endoscope devices.

7. A modular sterilizable and reusable introducer system as claimed: wherein said biopsy port is located directly opposite to the insufflations port and preferably extends from the hub side on a cylindrical shaped biopsy port body such that the longitudinal axes of the hub and the port body form a V shape to ensure that biopsy tool or other endoscopic devices can slide easily with minimal blockage into and out of cannula along with endoscope also inside cannula.

8. A modular sterilizable and reusable introducer system as claimed: said biopsy port comprising a sterilizable biopsy or other device fitting attachment means a serializable biopsy or other device fitting for operative connection at one end to said biopsy port through said sterilizable biopsy or other device fitting attachment means in a leak proof manner and at its other end to a cooperative biopsy means or other device in a leak proof manner.

9. A modular sterilizable and reusable introducer system as claimed: wherein endoscope fitting, insufflations fitting, cannula fitting, and biopsy fitting is a hydraulic or pneumatic fitting that mates with or couples to hydraulic or pneumatic fittings of endoscope, insufflation, cannula and biopsy/devices.

10. A modular sterilizable and reusable introducer system is claimed: wherein each said endoscope fitting, insufflations fitting, cannula fitting, and biopsy fitting comprise a rigid oblong hollow vessel with a connector end and an attachment end, the connector end adapted to mate with or couple to other hydraulic or pneumatic fittings/devices with corresponding connector ends to yield a leak proof connection between the two fittings and said attachment end connects to said endoscope fitting attachment means, insufflations fitting attachment means, cannula fitting attachment means and biopsy fitting attachment means respectively involving any type of leak proof fitting.

11. A modular sterilizable and reusable introducer system as claimed: wherein each said endoscope attachment means, said insufflations attachment means, said cannula attachment means and said biopsy attachment means are selected from any cooperative leak proof attachment including selected from threaded male-female attachment, socket attachment, compression ring attachment, luer type taper fitting.

12. A modular sterilizable and reusable introducer system as claimed: wherein said cannula comprise length of about 2 to 10 centimeters, outer diameter of about 0.3 to 3 millimeters, and wall thickness of about 0.03 to 0.10 millimeter and is obtained as semi-rigid cannula for a small degree of flexibility required to insert and navigate the cannula within the patient preferably the cannula can flex about 1-4 millimeters from end to end for this purpose.

13. A modular sterilizable and reusable introducer system as claimed: comprising said cannula is permanently attached or fixed to the hub wherein a proximal end of cannula is positioned around said cannula entry opening and permanently attached to cannula entry opening such as to contain without leaking the pressurized flow of gas or liquid from the interior of hub to the interior of cannula.

14. A modular sterilizable and reusable introducer system is claimed:
wherein said cannula cover comprises a retaining cylinder member that is a rigid cylindrical member with open ends, said retaining cylinder having an inner diameter that is sized to make a slip fit over the outside diameter of cannula, the retaining cylinder having a length that is at least as long as that of cannula, a wall thickness that is greater than that of cannula and capable of undergoing sterilization by autoclave without incurring deterioration, degradation, or failure.

15. A method of assembling and disassembling for use and reuse repeated times the modular sterilizable and reusable introducer system as claimed:
providing said sterilizable hub comprising a container or vessel body defining an internal hollow passage and having at least two ports comprising an endoscope port and an insufflations port, and having a cannula entry opening each said ports and cannula entry opening communicating with said internal hollow passage of said hub;
operatively connecting to said hub having permanently secured or releasable connected cannula or disconnecting after releasable connection selectively anyone or more of the following modular components involving:
operatively connecting said sterilizable endoscope fitting at one end to said endoscope port through said sterilizable endoscope fitting attachment means in a leak proof manner and at its other end to a cooperative releasable connectable endoscope means in a leak proof manner;
operatively connecting said sterilizable insufflation fitting at one end to said insufflation port through said sterilizable insufflation fitting attachment means in a leak proof manner and at its other end to a cooperative releasable connectable insufflations means in a leak proof manner; and
operatively connecting said sterilizable biopsy or other device fitting at one end to said biopsy port through said sterilizable biopsy or other device fitting attachment means in a leak proof manner and at its other end to a cooperative biopsy means or other device in a leak proof manner; such as to depending upon the use selectively connect or disconnect to and from said hub depending upon the stage of application of the relevant devices selected from endoscope, insufflations, biopsy or other such releasable connectable device.
